(19) United States
(12) Patent Application Publication
(10) Pub. No.: US 2015/0330893 A1
(43) Pub. Date: Nov. 19, 2015

(54) PHYSIOLOGICAL PARAMETER ANALYSIS ASSEMBLY

(71) Applicant: Daylight Solutions Inc., San Diego, CA (US)

(72) Inventors: Paul Larson, Poway, CA (US); William Chapman, San Diego, CA (US); Miles James Weida, Poway, CA (US)

(21) Appl. No.: 14/716,374

(22) Filed: May 19, 2015

Related U.S. Application Data
(60) Provisional application No. 62/000,411, filed on May 19, 2014.

(51) Int. Cl. G01N 21/3504; A61B 5/097; A61B 5/145; A61B 5/08

(52) U.S. Cl. CPC G01N 21/3504; A61B 5/082; A61B 5/097; A61B 5/14517; A61B 5/14546; G01N 2201/06113

(57) ABSTRACT

An analysis assembly (12) for analyzing one or more physiological parameters of a person (10) comprises a sensor assembly (14) and an analyzer (16). The sensor assembly (14) includes a sampler (218) that collects a sample (220) from the person (10); and a signal generating apparatus (222) that directs a mid-infrared light beam (232) toward the sample (220) and performs spectroscopy on the sample (220) to generate a signal (215) that is based at least in part on the one or more physiological parameters of the person (10). The sampler (218) and the signal generating apparatus (222) can be positioned less than approximately one meter from the person (10) while the sample (220) is being collected and spectroscopically scanned to generate the signal (215). The analyzer (16) receives and analyzes the signal (215) to determine the presence of the one or more physiological parameters in the sample (220).

### RELATED APPLICATION

This application claims priority on U.S. Provisional Application Ser. No. 62/000,411, filed May 19, 2014 and entitled “PHYSIOLOGICAL PARAMETER ANALYSIS ASSEMBLY”. As far as permitted, the contents of U.S. Provisional Application Ser. No. 62/000,411 are incorporated herein by reference.

### BACKGROUND

As the average person becomes more and more health-conscious, such person is typically more likely to participate in one or more exercise and/or rehabilitation programs in furtherance of any health-related goals. Additionally, athletes of today, who strive to improve their personal health and athletic performance, are also typically more likely to participate in such programs in furtherance of their athletic goals. Such exercise and/or rehabilitation programs can involve various exercises, medical treatments, nutritional programs, and anything else that can promote and/or enhance one's health and performance. In furtherance of such health-related and/or athletic-based goals, it is desired to exploit observables correlated to metabolic/physiological functions in order to tailor, refine, optimize or evaluate exercise regimens, programs, and/or treatments.

### SUMMARY

The present invention is directed toward an analysis assembly for analyzing one or more physiological parameters of a person during a specified period of time. In certain embodiments, the analysis assembly comprises a sensor assembly and an analyzer. The sensor assembly includes (i) a sampler that collects a sample from the person during the specified period of time; and (ii) a signal generating apparatus that directs a mid-infrared light beam toward the sample and performs spectroscopy on the sample to generate a signal that is based at least in part on the one or more physiological parameters of the person. Additionally, each of the sampler and the signal generating apparatus are positioned less than approximately one meter from the person while the sample is being collected and spectroscopically scanned to generate the signal. The analyzer receives the signal from the sensor assembly and analyzes the signal to determine the presence of the one or more physiological parameters in the sample. With this design, the analysis assembly is able to constantly monitor medical and health conditions, throughout the day, and including during exercise and fitness.

As provided herein, in various embodiments, it may be desired that the sampler of the sensor assembly be positioned in close proximity to the person, e.g., in close proximity to the mouth of the person, such that the samples captured by the sampler can be more accurately attributed to the person being evaluated. Additionally, it may also be desired to have the sensor assembly be incorporated within a portable device, such that each of the sampler and the signal generating apparatus are positioned in close proximity to the person, e.g., to the mouth of the person. By utilizing a portable device, the person is better able to utilize the analysis assembly in different locations and within different exercise scenarios.

In various embodiments, as described in detail herein, the analysis assembly senses, and analyzes and/or evaluates, one or more physiological parameters of the person during a specified period of time, e.g., when the person is engaging in an exercise routine, receiving medical treatments, ingesting nutritional supplements, etc. More specifically, the analysis assembly includes (i) the sensor assembly that senses the one or more physiological parameters of the person and generates a signal based on the sensed physiological parameters, and (ii) the analyzer that receives the signal related to the sensed physiological parameters, and analyzes and/or evaluates the received signal to determine the benefits that the person is receiving (or how the person is performing) during the specified period of time. Moreover, the sensor assembly can provide real-time measurement and feedback of various physiological parameters. Additionally, based on the results as determined by the analyzer, the person can tailor their exercise routines, medical treatments, and/or nutritional supplement ingestions to obtain the best overall results depending on the desired outcome.

In some embodiments, the sample comprises a breath sample from the person. In certain such embodiments, the sensor assembly is selectively coupled to the person, and the sampler is selectively positioned within less than approximately twenty centimeters from the mouth of the person. For example, in one embodiment, the analysis assembly can further comprise a headset that is selectively coupled to the person. The headset can include a coupling member that couples the headset to a head of the person, and an extension arm that is connected and extends away from the coupling member. In such embodiment, the sampler and the signal generating apparatus can be positioned at a distal end of the extension arm, with each of the sampler and the signal generating apparatus being positioned within less than approximately twenty centimeters from the mouth of the person.

Additionally, in certain embodiments, the sampler includes a sampler body, an intake that is coupled to the sampler body, and a pump that pumps the sample into the sampler body via the intake.

Further, in some embodiments, the signal generating apparatus includes a light source that emits the mid-infrared light beam that is directed toward the sample. In such embodiments, the mid-infrared light beam spectroscopically scans the sample to generate the signal. Moreover, the light source can be selectively adjustable to alternatively emit a first mid-infrared light beam having a first wavelength and a second mid-infrared light beam having a second wavelength that is different than the first wavelength.

Still further, in some such embodiments, the light source is a laser. For example, the light source can be a quantum cascade laser that emits the mid-infrared light beam that is directed toward the sample.

Additionally, in certain embodiments, the analyzer analyzes the signal to determine the presence of a first physiological parameter, and a second physiological parameter that is different than the first physiological parameter. In such embodiments, the analyzer can then determine a ratio of the first physiological parameter to the second physiological parameter. In certain such embodiments, the first physiological parameter includes a ketone, and the second physiological parameter includes carbon dioxide.

In one non-exclusive alternative embodiment, the sensor assembly can be coupled to an exercise apparatus.

In other embodiments, the sample comprises a sweat sample from the person. In some such embodiments, the sampler is adapted to be positioned in contact with the skin of the person to collect the sweat sample of the person, and the signal generating apparatus performs spectroscopy on the sweat sample to generate the signal. Additionally, in one such embodiment, the sampler is an Attenuated Total Reflectance window. In such embodiment, the Attenuated Total Reflectance window can include an inner surface that contacts the sample, and a non-planar, outer edge that is spaced apart from the sample. Moreover, in some embodiments, the Attenuated Total Reflectance window is formed from a material having a window refractive index that is higher than a sample refractive index of the sample. Further, in such embodiments, the analyzer can analyze the signal that was generated from the sweat sample for the presence of one or more of sodium, potassium, calcium, magnesium, lactate and urea.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified side view of a person engaging in an athletic activity and an embodiment of a physiological parameter analysis assembly having features of the present invention;

FIG. 2 is a simplified schematic illustration of the physiological parameter analysis assembly illustrated in FIG. 1;

FIG. 3 is a simplified side view of a person engaging in an athletic activity with the use of an exercise apparatus, and another embodiment of a physiological parameter analysis assembly having features of the present invention;

FIG. 4 is a simplified schematic illustration of the physiological parameter analysis assembly illustrated in FIG. 3;

FIG. 5 is a simplified side view of a person engaging in an athletic activity and still another embodiment of a physiological parameter analysis assembly having features of the present invention;

FIG. 6 is a simplified schematic illustration of a portion of the person and the physiological parameter analysis assembly illustrated in FIG. 5;

FIG. 7A is a simplified schematic illustration of a portion of an embodiment of a physiological parameter analysis assembly including a light beam that is transferred through a fiber for purposes of scanning a sample;

FIG. 7B is a simplified schematic illustration of a portion of another embodiment of a physiological parameter analysis assembly including a light beam that is transferred through a fiber for purposes of scanning a sample;

FIG. 7C is a simplified schematic illustration of a portion of still another embodiment of a physiological parameter analysis assembly including a light beam that is transferred through a fiber for purposes of scanning a sample; and

FIG. 7D is a simplified schematic illustration of a portion of yet another embodiment of a physiological parameter analysis assembly including a light beam that is transferred through a fiber for purposes of scanning a sample.

### DESCRIPTION

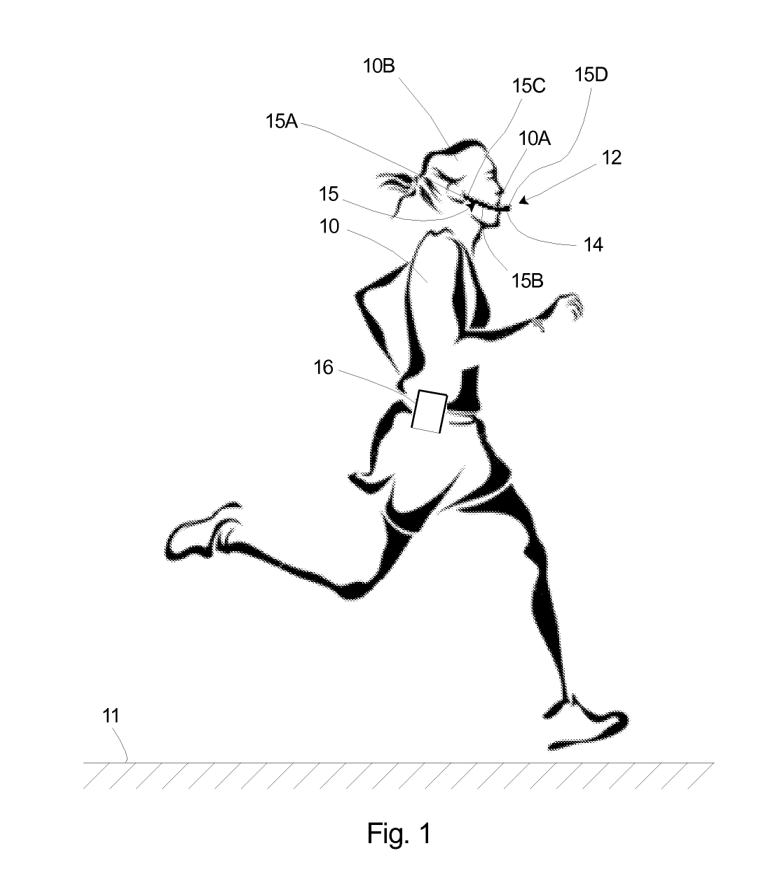

FIG. 1 is a simplified side view of a person 10 engaging in an athletic activity, e.g., running along a surface 11 such as the ground, treadmill, or a floor, and an embodiment of a physiological parameter analysis assembly 12 (also referred to herein simply as an “analysis assembly”) having features of the present invention. The design of the analysis assembly 12 can be varied as desired to suit the specific requirements of the person using the analysis assembly 12 and/or to suit the specific manner of use. In the embodiment illustrated in FIG. 1, the analysis assembly 12 includes a sensor assembly 14 that is selectively positionable relative to the person 10, and an analyzer 16.

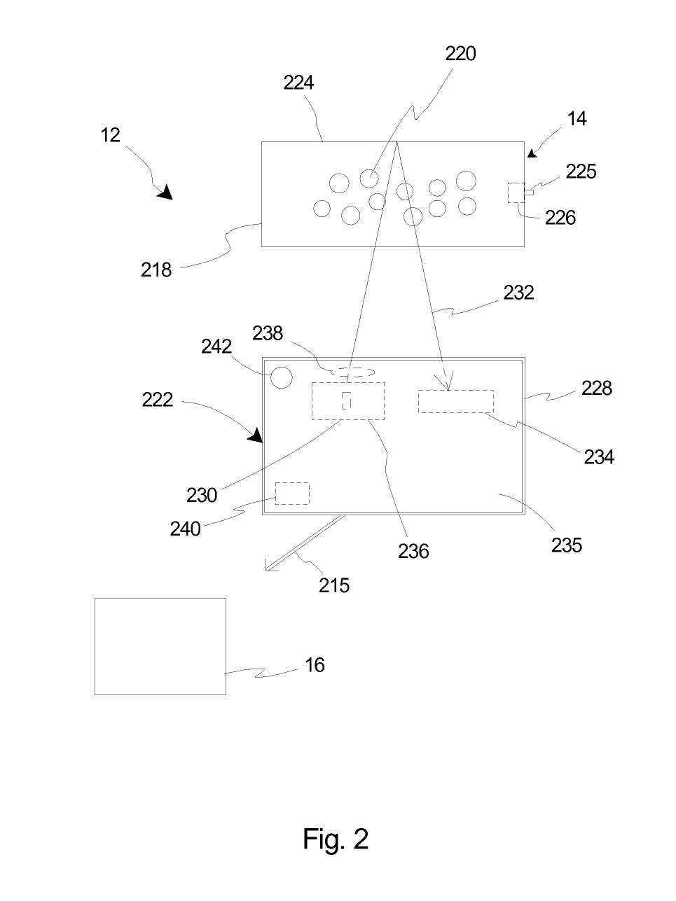

As an overview, the analysis assembly 12 is configured to sense, and analyze and/or evaluate, one or more physiological parameters of the person 10 during a specified period of time, e.g., when the person 10 is engaging in an exercise routine, receiving medical treatments, ingesting nutritional supplements, etc. More specifically, the analysis assembly 12 includes (i) the sensor assembly 14 that senses the one or more physiological parameters of the person 10, i.e. within one or more samples 220 (illustrated in FIG. 2) that are captured and/or collected from the person 10, and generates one or more signals 215 (illustrated in FIG. 2) and/or images based at least in part on the sensed physiological parameters in the samples 220; and (ii) the analyzer 16 that receives the one or more signals 215 and/or images related to the sensed physiological parameters from the samples 220, and analyzes and/or evaluates the received signals 215 and/or images to determine the benefits that the person 10 is receiving during the specified period of time based on the sensed physiological parameters. Additionally, based on the results as determined by the analyzer 16, the person can tailor their exercise routines, medical treatments, nutritional supplement ingestions, etc. to obtain the best overall results depending on the desired outcome. For example, in one non-exclusive application, as provided in greater detail herein below, the person 10 may be able to identify an ideal type and level of exercise to achieve maximum fat-burning results.

Alternatively, the analysis assembly 12 can be used to monitor medical and health conditions any time during the day.

As discussed herein, the particular physiological parameters that may be sensed, measured, analyzed and/or evaluated with the analysis assembly 12 can include certain indicator gases, e.g., ketones (which are produced when the body burns fat for energy or fuel), aldehydes, ammonia, etc., that are present within the one or more samples 220 that can be collected from the person 10. For example, in one specific non-exclusive alternative application, the analysis assembly 12 can focus on the detection and measurement of ketones, such as acetone, that can then be analyzed to determine the level of fat burning achieved during exercise.

Additionally, in some embodiments, the analysis assembly 12 can focus on ratios of certain specific physiological parameters that are present within the collected samples 220. For example, in one non-exclusive embodiment, the analysis assembly 12 can focus on ratios of the level of ketones within the collected samples 220 versus the level of carbon dioxide within the collected samples 220. In such embodiment, the samples 220 that are collected can comprise breath samples that are captured and/or collected during exhalations of the person 10, when such physiological parameters may be more prevalent within the samples 220. Moreover, such ratios can be determined at various times, e.g., during exercise, during treatments, etc., and compared to ratios that are established at other times, e.g., at rest, prior to exercise, after exercise, prior to treatments, after treatments, etc. In comparing such ratios at these different times, the analysis assembly 12 can then be better able to correlate any changes in ratios to the specific exercise routine, treatment programs, etc. in which the person is engaging. Additionally and/or alternatively, the measured, sensed and/or analyzed ratios determined during the specified periods of time can also be compared with population and/or demographic data that can be evaluated for potential for weight loss, fat burning, and other desired results.

Moreover, certain ratios between and/or among the various physiological parameters that may be sensed by the sensor assembly 14 are believed to provide indications of when the person 10 is in the perfect fat-burning zone, when the person 10 is better able to achieve weight loss, when the person 10 is gaining positive aerobic benefits, when the person 10 is gaining anaerobic benefits, when the person 10 is experiencing higher stress conditions, and/or when other appropriate factors are present. Thus, the feedback that can be provided with the use of the analysis assembly 12 can then be utilized to specifically tailor the exercise routine, the treatment program, etc. to achieve the desired results. Stated in another manner, the analysis assembly 12 can analyze the collected samples 220 to determine better, more effective exercise routines, treatment programs, etc. so that the person 10 can better achieve the desired health-related and/or athletic-based goals.

When conducting such a ratio-based analysis with the analysis assembly 12, it is initially desired to be specific about which indicators (i.e. physiological parameters such as ketones, carbon dioxide, aldehydes, etc.) are being monitored, to separate the specific indicators from one another, and to then quantify the chosen indicators with respect to one another. It should be appreciated that by simply focusing on ratios of any such indicators or physiological parameters that can be present in the collected samples, the analysis assembly 12 can utilize a lower level of precision as compared to an assembly that focuses only on absolute levels of such indicators or physiological parameters. Additionally and/or alternatively, in one embodiment, the analysis assembly 12 can be utilized to determine accurate absolute levels of any such indicators or physiological parameters during the specified periods of time.

As provided in detail herein, the analysis assembly 12 can collect samples 220 from the person 10 that are to be analyzed in various alternative manners. For example, depending on the particular design of the analysis assembly 12, the sample 220 that can be collected from the person 10 can be in the form of a breath sample (e.g., during exhalations), a sweat sample, a spatial (or zonal) sample, or another suitable sample that can be generated by the person 10 and/or captured near the person 10.

It should be appreciated that in order to more accurately capture samples 220 that only include physiological parameters related to the person 10 being evaluated, it may be necessary and/or desired that the samples 220 be captured within a certain proximity to the person 10. Additionally, as noted above, the sensor assembly 14 is selectively positionable relative to the person 10. For example, in various embodiments, it is desired that the sensor assembly 14 be positioned less than approximately one meter from the person 10, e.g., from a mouth 10A of the person 10 during collection of breath samples, such that the samples 220 captured by the sensor assembly 14 can be more accurately attributed to the person 10 being evaluated. Further, in other such non-exclusive alternative embodiments, it is desired that the sensor assembly 14 be positioned less than approximately fifty centimeters, thirty centimeters, twenty centimeters, ten centimeters, or five centimeters from the person 10, e.g., from the mouth 10A of the person 10. Additionally and/or alternatively, in certain embodiments, the sensor assembly 14 can be positioned so as to be in direct contact with the person 10, e.g., in contact with the skin of the person 10 during collection of sweat samples.

Further, as noted above, comparison samples can be collected before and/or after exercise, treatments, etc., which can be used in part to compensate for and/or take into consideration any ambient conditions that may exist in the area near where the person 10 being evaluated is located. For example, such comparison samples may compensate for and/or take into consideration other people who may be performing certain actions near the person 10 that can influence the physiological parameters being sensed.

As shown in FIG. 1, in certain embodiments, the sensor assembly 14 can comprise and/or incorporate a portable and “wearable” sensor, i.e. the sensor assembly 14 can be selectively coupled to the person 10, that provides real-time measurement and feedback of various physiological parameters. More particularly, in this embodiment, the sensor assembly 14 can be provided in the form of a headset 15 that can be selectively coupled to the person 10, e.g., to a head 10B of the person 10. As shown, in one embodiment, the headset 15 can include a coupling member 15A, e.g., an earpiece or a clip over a portion of the head, for coupling the headset 15 to a head 10B of the person 10, and a boom microphone-type, extension arm 15B that is connected to and extends away from the coupling member 15A to near the head 10B (more specifically the mouth 10A) of the person 10. More particularly, the extension arm 15B can include a proximal end 15C that is connected to the coupling member 15A, and a distal end 15D that is positioned in close proximity to the mouth 10A of the person 10. Additionally, the distal end 15D of the extension arm 15B can include and/or incorporate the sensor assembly 14 so that the sensor assembly 14 is effectively positioned near the mouth 10A of the person 10 and collects breath samples from the person 10 during the relevant periods of time. Stated in another manner, the sensor assembly 14 can be selectively coupled to the distal end 15D of the extension arm 15B so that the sensor assembly 14 is effectively positioned near the mouth 10A of the person 10 and collects breath samples from the person 10 during the relevant periods of time.

In this case, the wearable boom can also serve as a single cell or multi-pass cell to capture breath exhalations as a desired sample 220. The selected indicators and/or physiological parameters can then be measured, sensed, evaluated and/or analyzed for their presence within the breath sample. The multi-pass cell option can provide improved detection sensitivity by increasing the total optical path length that travels through the sample 220.

It should also be appreciated that the interface between the person 10 and the analysis assembly 12 and/or the means of delivery of the sample 220 from the person 10 to the analysis assembly 12 can be varied depending on the type of sample 220 that is being generated and/or captured.

Additionally, as shown in FIG. 1, in certain embodiments, the analyzer 16 can be wirelessly connected to the sensor assembly 14. In one such embodiment, the analyzer 16 can be incorporated within an application of a smart phone. In particular, in such embodiment, the sensor assembly 14 can utilize a Bluetooth interface to a smart phone app for real-time feedback of physiological parameters present in the breath, sweat and/or space of the person 10, i.e. who is engaging in an exercise routine, treatment program, etc. In another such embodiment, the analyzer 16 can be incorporated within a computer that is wirelessly connected to the sensor assembly 14. Alternatively, the analyzer 16 can be included in another appropriate format, provided that the analyzer 16 has the ability to receive and analyze the physiological parameters that are sensed and/or the signals 215 that are generated within the sensor assembly 14. Still alternatively, in one non-exclusive alternative embodiment, the analyzer 16 can have a wired connection to the sensor assembly 14.

Turning now to FIG. 2, this Figure is a simplified schematic illustration of the analysis assembly 12, i.e. the sensor assembly 14 and the analyzer 16, illustrated in FIG. 1.

The design of the sensor assembly 14 can be varied depending on the requirements of the analysis assembly 12. In this embodiment, the sensor assembly 14 includes a sampler 218 (or intake) that collects the desired sample 220 (illustrated as a plurality of small circles) from the person 10 (illustrated in FIG. 1) during the desired periods of time; and a signal generating apparatus 222 that performs spectroscopy on the collected sample 220 to generate a signal 215 that is sent to and subsequently received and analyzed by the analyzer 16. As provided herein, the signal 215 can be based at least in part on one or more physiological parameters that exist within the collected sample 220.

As noted above, in various embodiments, it may be desired that the sensor assembly 14 be positioned in close proximity to the person 10, e.g., to the mouth 10A of the person 10, such that the samples 220 captured by the sensor assembly 14 can be more accurately attributed to the person 10 being evaluated. More particularly, in such embodiments, it may be desired that each of the sampler 218 and the signal generating apparatus 222 be positioned in close proximity to the person 10. For example, in certain non-exclusive alternative embodiments, it is desired that the sensor assembly 14, i.e. each of the sampler 218 and the signal generating assembly 222, be positioned less than approximately one meter, fifty centimeters, thirty centimeters, twenty centimeters, ten centimeters, or five centimeters from the person 10, e.g., from the mouth 10A of the person 10. Additionally and/or alternatively, in certain embodiments, the sensor assembly 14 can be positioned so as to be in direct contact with the person 10. With this design, not only the collection of the sample 220, but also the spectroscopy that is performed on the sample 220, will occur in close proximity to the person 10, e.g., less than approximately one meter from the person 10. Alternatively, the sensor assembly 14, i.e. one or both of the sampler 218 and the signal generating assembly 222, may be positioned greater than approximately one meter from the person 10.

Additionally, as noted, the sampler 218 is configured to collect one or more samples 220 from the person 10 during the desired and/or specified periods of time. The design of the sampler 218 can be varied depending on the particular requirements of the analysis assembly 12 and the type of sample 220 to be collected. For example, in the embodiment illustrated in FIG. 2, the sampler 218 is configured to collect one or more breath or spatial samples 220 from the person 10. More particularly, in such embodiment, the sampler 218 can include a sampler body 224 (e.g., a single cell or multi-pass cell), and an intake tube 225 (also referred to simply as an “intake”) that is coupled to the sampler body 224. Further, the sampler 218 can also include a small pump 226 (illustrated in phantom) that pumps the sample 220 into the sampler body 224 via the intake 225. With this design, the sampler 218 effectively sips the air near and/or around the mouth 10A (illustrated in FIG. 1) of the person 10 to collect the desired sample 220 on which spectroscopy is subsequently performed by the signal generating apparatus 222. Additionally and/or alternatively, the sampler 218 need not be positioned directly near the mouth 10A of the person 10, as the sampler 218 can collect the desired samples 220 by being positioned in the general area or space of the person 10. Still alternatively, the sampler 218 can have another suitable design. For example, the sampler 218 can simply use pressure from the exhalations from the person 10 that is blown into, near and/or through the sampler 218, i.e. without the need for a pump.

In certain applications, the sampler 218 primarily collects the desired samples 220 during exhalations of the person 10, as such periods typically would be able to provide more data for analyzing the desired correlations between and/or among the specified physiological parameters. For example, in one application, the sensor assembly 14 can look at and/or focus on the pace of breathing of the person 10 with respect to spikes in carbon dioxide that are present in the samples 220, which can be used to establish a time signature for the sampling procedure. Subsequently, the analysis assembly 12, i.e. the analyzer 16, can analyze the generated data, i.e. via captured images or other generated signals 215, to determine what other physiological parameters are correlated with that time signature. Thus, the levels of the other physiological parameters found in the generated data can be effectively compared and correlated with the level of carbon dioxide seen in the individual exhalations.

Additionally, as provided above, the signal generating apparatus 222 performs spectroscopy on the collected sample 220 so as to generate a signal 215 that is sent to and subsequently received and analyzed by the analyzer 16. Stated in another manner, in various embodiments, as illustrated in FIG. 2, the signal generating apparatus 222 can capture images and/or detect features and aspects of one or more points of the sample 220 that can be transferred as signals, e.g., image signals, to the analyzer 16 for purposes of analysis.

The design of the signal generating apparatus 222 can be varied to suit the specific requirements of the analysis assembly 12 and/or the type of sample 220 that is being collected and analyzed. For example, in some embodiments, as shown in FIG. 2, the signal generating apparatus 222 can include an apparatus frame 228, a light source 230 (illustrated in phantom) that emits a light beam 232 (shown partially in phantom) that is directed toward the sample 220, and a detector 234 (illustrated in phantom). Alternatively, the signal generating apparatus 222 can include more components or fewer components than those specifically illustrated in FIG. 2.

The apparatus frame 228 can be rigid and can support at least some of the other components of the signal generating apparatus 222. In one embodiment, the apparatus frame 228 includes a generally rectangular shaped hollow body that forms a cavity 235 that receives and retains at least some of the other components of the signal generating apparatus 222. Alternatively, the apparatus frame 228 can have a different design and/or a different shape.

The light source 230 generates and/or emits the light beam 232 that is directed toward the sample 220 that has been collected by the sampler 218. More particularly, once the light source 230 has emitted the light beam 232, the light beam 232 is directed toward the sample 220 so that the sample 220 may be properly and effectively illuminated by the light beam 232. Additionally, the light source 230 generates and/or emits the light beam 232 that can be used to scan the sample 220 that has been collected by the sampler 218 for purposes of analysis. For example, in certain embodiments, as provided in greater detail herein below, the light source 230 can utilize tunable laser radiation to spectroscopically interrogate the sample 220 in order to analyze and identify the physiological parameters that are present in the sample 220.

The design of the light source 230 can be varied as desired so as to emit the desired light beam 232. In certain embodiments, the light source 230 is a laser. For example, the light source 230 can include a mid-infrared (MIR) laser source that can be either a fixed wavelength or a selectively tunable laser source so as to generate and/or emit a narrow linewidth, accurately settable MIR beam as the light beam 232. Stated in another manner, the light source 230 can be a mid-infrared laser source, and the light beam 232 can be a mid-infrared beam, i.e. a light beam having a selectively tunable wavelength of between approximately 3.0 micrometers and 12.0 micrometers, that is generated and/or emitted by the mid-infrared laser source. In one embodiment, the light source 230 can be a single emitter infrared semiconductor laser. Moreover, in alternative embodiments, the light source 230 can be a pulsed laser, i.e. which requires less power and generates less heat, and/or a continuous wave (CW) laser.

Additionally, in one such embodiment, the light source 230 can be a quantum cascade laser (QCL) that generates and/or emits a coherent light beam 232. More particularly, in such embodiment, the light source 230 can include a gain medium 236, e.g., a Quantum Cascade (QC) gain medium, that directly emits the light beam 232 that is in the mid-wavelength infrared range without any frequency conversion. With this design, electrons transmitted through the QC gain medium 236 emit one photon at each of the energy steps. For example, the QC gain medium 236 can use two different semiconductor materials such as InGaAs and AlInAs (grown on an InP or GaSb substrate, for example) to form a series of potential wells and barriers for electron transitions. The thickness of these wells/barriers determines the wavelength characteristic of the QC gain medium 236. Additionally, in one, non-exclusive such embodiment, the semiconductor QCL laser chip is mounted epitaxial growth side down. Alternatively, the light source 230 can include an interband-cascade (IC) laser, a diode laser, or any other laser capable of generating radiation in the appropriate mid-wavelength infrared spectral region. Still alternatively, the light source 230 can be another suitable light source that generates and/or emits an alternatively suitable light beam.

Further, the light source 230 can also include an adjustment assembly (not illustrated) that can be utilized to precisely select and adjust the wavelength of the light beams 232 that are emitted from the light source 230. For example, in one non-exclusive embodiment, the adjustment assembly can include a diffraction grating (not illustrated) and a grating mover (not illustrated) that selectively moves, e.g., rotates, the diffraction grating to adjust the wavelength of the light beam 232. The diffraction grating can be continuously monitored with an encoder (not illustrated) that provides closed-loop control of the grating mover. With this design, the wavelength of the light beam 232 can be selectively adjusted in a closed-loop fashion so that the sample 220 can be analyzed at many different, precise, selectively adjustable wavelengths through a portion of or the entire MIR spectrum. A non-exclusive example of a suitable light source 230 is provided in U.S. Pat. No. 7,848,382, entitled “LASER SOURCE THAT GENERATES A PLURALITY OF ALTERNATIVE WAVELENGTH OUTPUT BEAMS”.

As far as permitted, the contents of U.S. Pat. No. 7,848,382 are incorporated herein by reference. Alternatively, the adjustment assembly can include a MEMs grating, a tunable filter, or another suitable mechanism to precisely select and adjust the wavelength of the light beams 232 that are emitted from the light source 230.

It should be appreciated that different physiological parameters in the sample 220 are more apparent and/or more identifiable when scanned by light beams of different wavelengths. For example, (i) a first physiological parameter may be more apparent and/or identifiable when scanned by a light beam of a first wavelength; (ii) a second physiological parameter may be more apparent and/or identifiable when scanned by a light beam of a second wavelength that is different than the first wavelength; and (iii) a third physiological parameter may be more apparent and/or identifiable when scanned by a light beam of a third wavelength that is different than the first wavelength and the second wavelength. Thus, by utilizing a light source 230 that enables the selective tuning of the light beam 232 that is generated and/or emitted by the light source 230, the light source 230 can be utilized for different spectroscopic applications, i.e. to identify specific alternative physiological parameters that may be present in the sample 220. Accordingly, depending on the particular physiological parameters, e.g., carbon dioxide, ketones, aldehydes, etc., that are being focused on by the user of the analysis assembly 12, the light source 230 can be selectively tuned to the appropriate wavelength for more accurately and precisely identifying such physiological parameters within the sample 220.

The detector 234 senses and/or captures rays generated from the light beam 232 from the light source 230 scanning the sample 220, and converts the rays into an array of electrical signals. In the non-exclusive embodiment illustrated in FIG. 2, the rays are reflected off of the sampler 218. Additionally, the electrical signals can be used to generate one or more optical images, i.e. optical signals, that represent an image of the sample 220. As non-exclusive examples, the detector 234 can include a point detector, one or more elements that measure intensity, a photodiode, or another type of sensor. Alternatively, the detector 234 can include another type of sensor.

In one embodiment, the detector 234 can include a two-dimensional array of photosensitive elements (pixels) that are sensitive to the wavelength of the light beam 232. For example, if the light beam 232 in the MIR range, the detector 234 can be an MIR imager. More specifically, if the light beam 232 in the infrared spectral region from between approximately 3.0 micrometers and 12.0 micrometers, the detector 234 is sensitive to the infrared spectral region from between approximately 3.0 micrometers and 12.0 micrometers.

Additionally, in one non-exclusive alternative embodiment, the signal generating apparatus 222 can be and/or include an image capturing device, e.g., an infrared camera, that captures images of the sample 220 that can be transferred as signals 215, e.g., image signals, to the analyzer 16 for purposes of analysis.

It should be appreciated that the light beam 232 can be utilized for purposes of spectroscopic analysis of the sample 220 in a single-pass or multi-pass through the sampler 218, and such use of the light beam 232 is not limited to the specific usage shown in the schematic illustration of FIG. 2.

Further, in some embodiments, the electrical signals and/or optical signals can then be wirelessly sent to the analyzer 16 so that the desired analysis can be undertaken. For example, the analyzer 16 can detect and/or analyze any physiological parameters that may be present in the signals depending upon the particular wavelength of the light beam 232 within the infrared spectral region from between approximately 3.0 micrometers and 12.0 micrometers. Moreover, the analyzer 16 can provide any such analytical or diagnostic information in a linear, tabular or graphic format.

Additionally, it should be appreciated that the sensor assembly 14 can include additional features that better enable the sensor assembly 14 to function as desired. For example, in some embodiments, the sensor assembly 14 can further include an optical assembly 238 (illustrated in phantom), a power source 240 (illustrated in phantom), and a switch 242. In some embodiments, the optical assembly 238 can include one or more lenses, mirrors and/or other optical elements that work in conjunction with one another to enable any desired focusing, shaping and directing of the light beam 232 from the light source 230 toward the sample 220, and/or to focus the light or optical image onto the detector 234. Further, the power source 240, e.g., one or more batteries for use in a portable analysis assembly 12, can provide the necessary and desired power to effectively and efficiently operate the sensor assembly 14. For example, the power source 240 can enable a user to selectively activate and control the sampler 218 and the light source 230. Still further, the switch 242 can enable the user to selectively turn the sensor assembly 14 on and off as desired, to selectively adjust the wavelength of the light beam 232, and/or to control other features and elements of the sensor assembly 14.

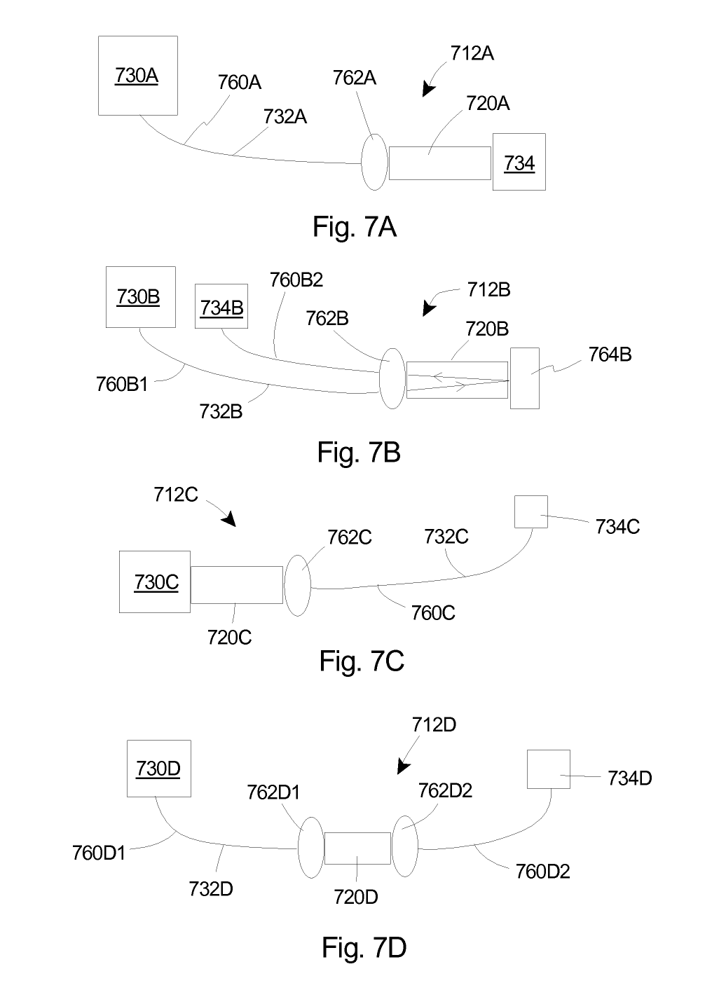

Further, it should also be appreciated that the sample 220 can be collected in a slightly different manner from what is specifically illustrated in FIG. 2. For example, in certain embodiments, the light beam 232 from the light source 230, e.g., the MIR laser source, can be directed through a fiber, e.g., an MIR optical fiber, and through one or more lenses before and/or after the light beam 232 is utilized to spectroscopically analyze the sample 220. Additionally, additional fibers and/or lenses can be utilized for transmitting and/or directing the light beam 232 before any rays generated from the light beam 232 from the light source 230 scanning the sample 220 are sensed and/or captured by the detector 234. FIGS. 7A-7D are simplified schematic illustrations of some non-exclusive alternative such embodiments.

For example, FIG. 7A is a simplified schematic illustration of a portion of an embodiment of a physiological parameter analysis assembly 712A including a light beam 732A, i.e. an MIR laser beam, from a light source 730A, i.e. an MIR laser source, that is transmitted through a fiber 760A, i.e. an MIR optical fiber, for purposes of scanning a sample region 720A. In certain applications, the sample region 720A can be an open space that includes breath or spatial samples from the person 10 (illustrated in FIG. 1) being evaluated. More particularly, the light beam 732A is transmitted via the fiber 760A that is coupled to the light source 730A through a lens 762A before being utilized to spectroscopically analyze the sample region 720A. The rays generated from the light beam 732A scanning the sample region 720A are subsequently sensed and/or captured by a detector 734A in this single-pass arrangement.

Additionally, FIG. 7B is a simplified schematic illustration of a portion of another embodiment of a physiological parameter analysis assembly 712B including a light beam 732B, i.e. an MIR laser beam, from a light source 730B, i.e. an MIR laser source, that is transmitted through a first fiber 760B1, i.e. a first MIR optical fiber, for purposes of scanning a sample region 720B. More particularly, the light beam 732B is directed via the first fiber 760B1 that is coupled to the light source 730B through a lens 762B before being utilized to spectroscopically analyze the sample region 720B. The rays generated from the light beam 732A scanning the sample region 720A are subsequently reflected off a reflector 764B back through the lens 762B and are transmitted by a second fiber 760B2, i.e. a second MIR optical fiber, before being sensed and/or captured by a detector 734B in this double-pass arrangement.

Further, FIG. 7C is a simplified schematic illustration of a portion of another embodiment of a physiological parameter analysis assembly 712C. In this embodiment, a light beam 732C, i.e. an MIR laser beam, from a light source 730C, i.e. an MIR laser source, spectroscopically analyzes a sample region 720C before being directed through a lens 762C and transmitted via a fiber 760C, an MIR optical fiber, before being sensed and/or captured by a detector 734C.

Still further, FIG. 7D is a simplified schematic illustration of a portion of still another embodiment of a physiological parameter analysis assembly 712D. In this embodiment, a light beam 732D, i.e. an MIR laser beam, from a light source 730D, an MIR laser source, is transmitted through a first fiber 760D1, i.e. a first MIR optical fiber, for purposes of scanning a sample region 720D. More particularly, the light beam 732D is transmitted via the first fiber 760D1 that is coupled to the light source 730D through a first lens 762D1 before being utilized to spectroscopically analyze the sample region 720D. Subsequently, rays generated from the light beam 732D scanning the sample region 720D are subsequently directed through a second lens 762D2 and are transmitted through a second fiber 760D2, i.e. a second MIR optical fiber, before being sensed and/or captured by a detector 734D.

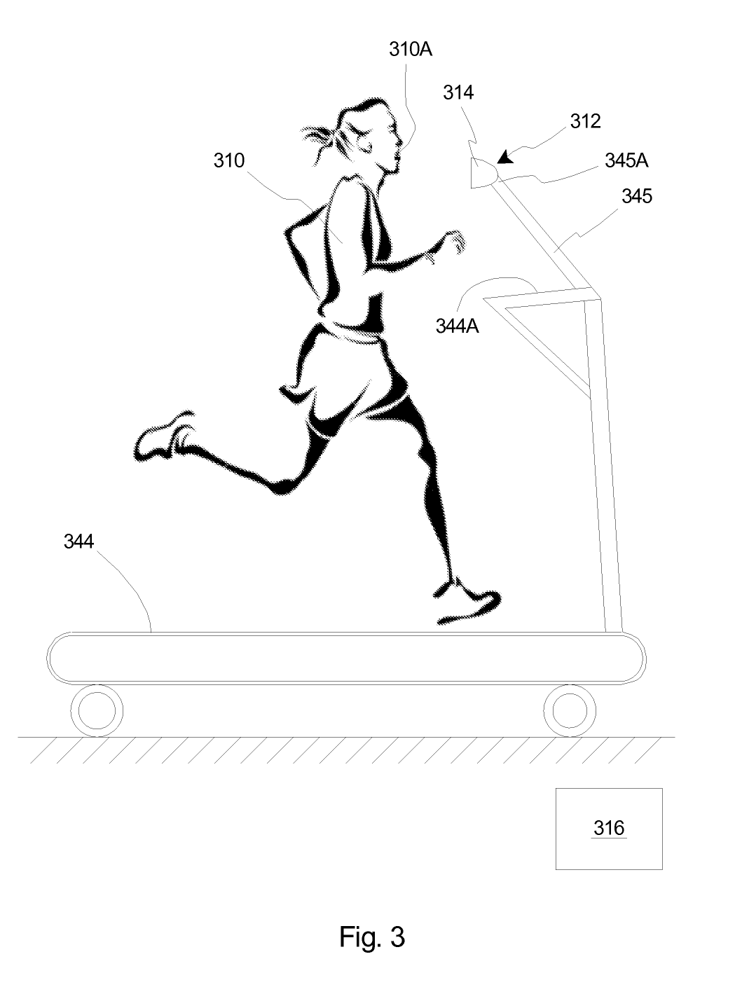

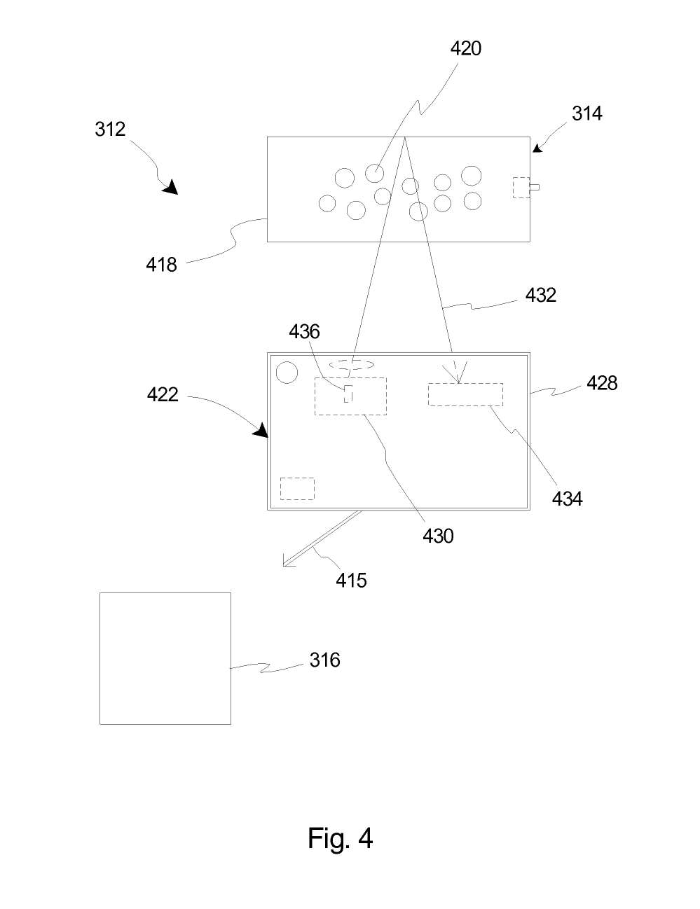

Returning now to FIG. 3, this Figure is a simplified side view of a person 310 engaging in an athletic activity with the use of an exercise apparatus 344, and another embodiment of an analysis assembly 312 having features of the present invention. The analysis assembly 312 in this embodiment is somewhat similar to the analysis assembly 12 illustrated and described above in relation to FIG. 1. More particularly, the analysis assembly 312 again includes a sensor assembly 314 and an analyzer 316 that are somewhat similar to the sensor assembly 14 and the analyzer 16 illustrated and described above in relation to FIG. 1. For example, as with the previous embodiment, the sensor assembly 314 again is configured to sense one or more physiological parameters of the person 310 during specified periods of time. Additionally, the analyzer 316 again receives one or more signals 415 (illustrated in FIG. 4) and/or images related to the sensed physiological parameters, and analyzes and/or evaluates the received signals 415 and/or images to determine the benefits that the person 310 is receiving during the specified periods of time.

However, in this embodiment, the sensor assembly 314 is coupled to the exercise apparatus 344, e.g., a treadmill, a stationary bicycle, a rowing machine, an elliptical trainer, a stair stepper, etc. In the embodiment shown in FIG. 3, the sensor assembly 314 can be adjustably coupled to and/or integrated into the exercise apparatus 344 such that the sensor assembly 314, i.e. a sampler 418 (illustrated in FIG. 4) and a signal generating apparatus 422 (illustrated in FIG. 4), can be positioned in close proximity to, e.g., less than approximately one meter from, the face and/or mouth 310A of the person 310 who is utilizing the exercise apparatus 344. By positioning the sensor assembly 314 in close proximity to the face and/or mouth 310A of the person 310 utilizing the exercise apparatus 344, the sensor assembly 314 is able to sense the one or more physiological parameters of the person 310, e.g., within collected breath samples, during a specified period of time in a manner substantially similar to the previous embodiment.

For example, in one embodiment, the exercise apparatus 344 can include a flexible, extension arm 345 that is coupled to a control panel 344A (or another portion of the exercise apparatus 344). Additionally, the sensor assembly 314 can be coupled to and/or incorporated within the flexible, extension arm 345. More particularly, the sensor assembly 314, i.e. the sampler 418 and the signal generating apparatus 422, can be coupled to and/or incorporated within a distal end 345A of the extension arm 345 away from the control panel 344A. With this design, the sampler 418 and the signal generating apparatus 422 can be adjustably and selectively positioned in close proximity to the mouth 310A of the person 310 using the exercise apparatus 344 to effectively collect and spectroscopically analyze breath samples from the person 310 during the relevant periods of time. Stated in another fashion, the extension arm 345 can be rotated and/or extended to move the sampler 418 and signal generating apparatus 422 to be closer to the person 310. For example, in different embodiments, the sampler 418 and the signal generating apparatus 422 can be selectively positioned less than approximately one meter, fifty centimeters, thirty centimeters, twenty centimeters, ten centimeters, or five centimeters from the mouth 310A of the person 310.

Additionally, as noted, the analyzer 316 again receives, analyzes and evaluates the one or more signals 415 related to the sensed physiological parameters to determine the benefits that the person 310 is receiving during the specified periods of time. Further, the analyzer 316 can again be wirelessly connected to the sensor assembly 314 in the form of an application of a smart phone, an application within a computer, and/or in another appropriate format, provided that the analyzer 316 has the ability to receive and analyze the physiological parameters that are sensed and/or the signals 415 or images that are generated within the sensor assembly 314.

FIG. 4 is a simplified schematic illustration of the analysis assembly 312, i.e. the sensor assembly 314 and the analyzer 316, illustrated in FIG. 3. In this embodiment, the analysis assembly 312 is substantially similar to the analysis assembly 12 illustrated and described above in relation to FIGS. 1 and 2. More particularly, the analysis assembly 312 again includes the sensor assembly 314 and the analyzer 316 that are substantially similar to the sensor assembly 14 and the analyzer 16 illustrated and described above in relation to FIGS. 1 and 2.

As with the previous embodiment, the sensor assembly 314 again includes (i) a sampler 418 (or intake) that collects a desired sample 420 (illustrated as a plurality of small circles) from the person 310 (illustrated in FIG. 3) during the desired periods of time; and (ii) a signal generating apparatus 422 that performs spectroscopy on the collected sample 420 to generate a signal 415 that is sent to and subsequently received and analyzed by the analyzer 316. In one embodiment, the generated signal 415 can again be wirelessly sent to the analyzer 316 so that the desired analysis can be undertaken.

Additionally, as noted above, the sampler 418 can be positioned near the face and/or mouth 310A (illustrated in FIG. 3) of the person 310 utilizing the exercise apparatus 344 (illustrated in FIG. 3). As such, the sampler 418 can be utilized to collect one or more breath or spatial samples 420 from the person 310. Alternatively, the sampler 418 can have a different design and/or can be positioned in a different manner.

Further, in certain embodiments, the signal generating apparatus 422 can again include an apparatus frame 428, a light source 430, and a detector 434 (illustrated in phantom) that captures images of the sample 420 that can be transferred as signals 415, e.g., image signals, to the analyzer 316 for purposes of analysis. Additionally, such components of the signal generating apparatus 422 can be substantially similar to the elements illustrated and described above in relation to FIG. 2. Accordingly, such elements will not be described in substantial detail herein.

As above, the light source 430 generates and/or emits the light beam 432 (shown partially in phantom) that is directed toward the sample 420 that has been collected by the sampler 418. The light source 430 can thus generate and/or emit the light beam 432 that can be used to spectroscopically scan the sample 420 that has been collected by the sampler 418 for purposes of analysis. Moreover, in certain embodiments, the light source 430 can again utilize tunable laser radiation to spectroscopically interrogate the sample 420 in order to analyze and identify the physiological parameters that are present in the sample 420. For example, in some such embodiments, the light source 430 can again be an MIR light source that can be selectively tuned so as to generate and/or emit a narrow linewidth, accurately settable MIR beam as the light beam 432. In one such embodiment, the light source 430 can be a quantum cascade laser (QCL), which includes a Quantum Cascade (QC) gain medium 436 that directly emits the light beam 432 that is in the mid-wavelength infrared range without any frequency conversion. Alternatively, the light source 430 can include an interband-cascade (IC) laser, a diode laser, or any other laser capable of generating radiation in the appropriate mid-wavelength infrared spectral region. Still alternatively, the light source 430 can be another suitable light source that generates and/or emits an alternatively suitable light beam.

Additionally, as with the previous embodiment, it should be appreciated that the light beam 432 can be utilized for purposes of spectroscopic analysis of the sample 420 in a single-pass or multi-pass through the sampler 418, and such use of the light beam 432 is not limited to the specific usage shown in the schematic illustration of FIG. 4.

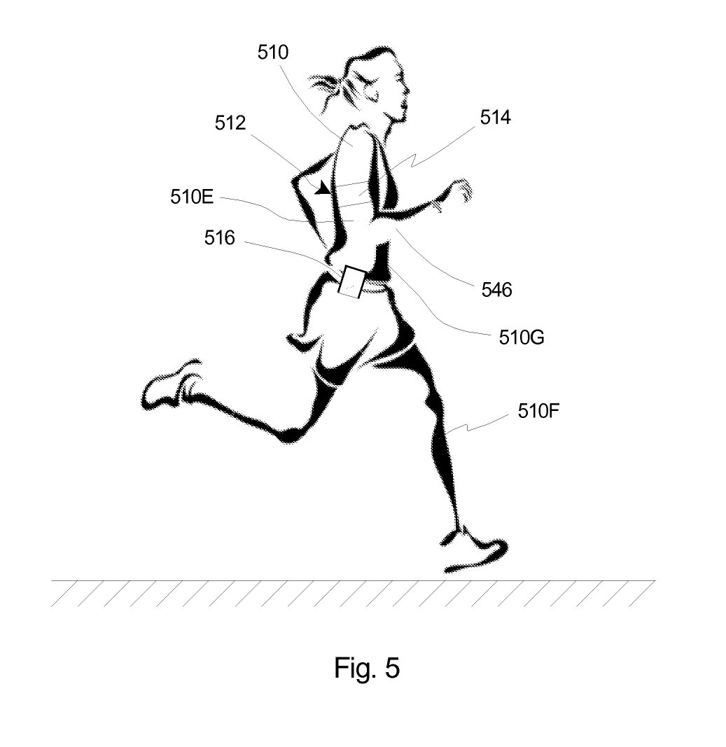

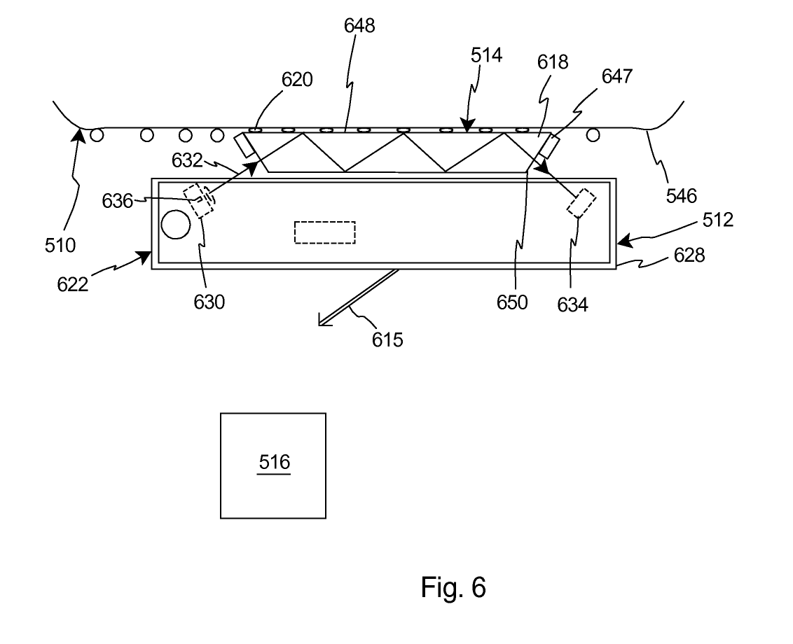

FIG. 5 is a simplified side view of a person 510 engaging in an athletic activity, e.g., running, and still another embodiment of an analysis assembly 512 having features of the present invention. The analysis assembly 512 in this embodiment is somewhat similar to the analysis assemblies 12, 312 illustrated and described above. More particularly, in this embodiment, the analysis assembly 512 again includes (i) a sensor assembly 514 that senses the one or more physiological parameters of the person 510 during a specified period of time; and (ii) an analyzer 516 that receives one or more signals 615 (illustrated in FIG. 6) and/or images related to the sensed physiological parameters, and analyzes and/or evaluates the received signals 615 and/or images to determine the benefits that the person 510 is receiving during the specified period of time. However, in this embodiment, the sensor assembly 514 has a different design and functions in a different manner as compared to the previous embodiments.

As shown in FIG. 5, the sensor assembly 514 can be a portable device that can be selectively attached to the person 510, e.g., to an arm 510E, a leg 510F, a torso 510G, or other appropriate area of the person 510, in order to effectively sense and/or measure the one or more physiological parameters of the person 510. As described in greater detail herein below, in this embodiment, the sensor assembly 514 utilizes a mid-infrared, attenuated total reflectance (ATR) based sensor to sense, detect and/or measure various physiological parameters of the person 510 through direct contact with the skin 546 of the person 510. For example, the sensor assembly 514 can include a wearable contact sensor that utilizes an ATR method and Bluetooth interface to a smart phone app, a computer, or other suitable analyzer 516 for real-time feedback of physiological parameters extractable through the skin 546.

Further, as opposed to the previous embodiments, which analyzed the selected indicators and/or physiological parameters present in the breath or area of the person; in this embodiment, the analysis assembly 512 is utilized to analyze the selected indicators and/or physiological parameters present in the sweat of the person 510. For example, the sweat sample of the person 510 can include water, minerals (e.g., sodium, potassium, calcium and magnesium), lactate and urea, one or more of which can be specifically identified by the sensor assembly 514. Thus, the values of such substances in the sweat sample of the person 510 can be analyzed and correlated, and ratios established, which can be utilized to determine any benefits that the person 510 may be gaining through participation in the chosen athletic activity.

FIG. 6 is a simplified schematic illustration of a portion of the person 510, i.e. the skin 546 of the person 510, and the analysis assembly 512, i.e. the sensor assembly 514 and the analyzer 516, illustrated in FIG. 5. In this embodiment, the analysis assembly 512 has some features in common as compared to the previous embodiments. For example, the analysis assembly 512 again includes a sampler 618 that collects, captures and/or contacts a desired sample 620 (illustrated as a plurality of small circles and/or ovals) from the person 510 during the desired periods of time; and a signal generating apparatus 622 that performs spectroscopy on the collected sample 620 to generate a signal 615 that is sent to and subsequently received and analyzed by the analyzer 516. In one embodiment, the generated signal 615 can again be wirelessly sent to the analyzer 516 so that the desired analysis can be undertaken.

However, in this embodiment, the sensor assembly 514 is different in design and function as compared to the previous embodiments. More particularly, the sampler 618 is different in design and function, and the sampler 618 collects and/or contacts a different type of sample 620, i.e. the sweat of the person 510 (a sweat sample). Additionally, although the signal generating apparatus 622 has certain features in common with the previous embodiments, the signal generating apparatus 622 interacts with the collected sample 620 in a different manner.

The design of the sampler 618 can be varied to suit the specific requirements of the analysis assembly 512. In this embodiment, the sampler 618 is positioned substantially directly adjacent to the skin 546 of the person 510 so that the sampler 618 comes in direct contact with the sample 620, i.e. the sweat, from the person 510. In one embodiment, the sampler 618 is an ATR crystal (also referred to herein generally as an “ATR window”) that directly contacts the sweat 620 of the person 510.

Additionally, in some embodiments, the sampler 618 can be used in conjunction with and/or incorporate one or more wicking members 647 (or other type of sample flushing system). As shown, the wicking members 647 can be positioned substantially adjacent to the sampler 618 and/or between a portion of the sampler 618 and the skin 546 of the person 510. The wicking members 647 are positioned to gradually draw the sweat sample 620 outwardly away from being captured between the sampler 618 and the skin 546 of the person 510. With this design, the old sweat samples 620 that may have already been spectroscopically analyzed can be gradually changed (or drawn) out, with new sweat samples 620 taken their place in the area between the ATR window 618 and the skin 546 of the person 510. With this continual changing of the sweat samples 620 between the ATR window 618 and the skin 546 of the person 510, the old sweat samples 620 will be inhibited from impact the analysis of the new sweat samples 620. Stated in another manner, with this design, any physiological parameters that may have been detected in the old sweat samples 620 will have been removed from the area of spectroscopic analysis so as to inhibit such physiological parameters from potentially adversely impacting the spectroscopic analysis of the new sweat samples 620.

It should be noted that in certain embodiments, the wicking members 647 can be selectively attached and detached from the sampler 618 so that replacement wicking members 647 can be utilized.

Further, as noted above, the signal generating apparatus 622 has certain features in common with the previous embodiments. For example, the signal generating apparatus 622 can again include an apparatus frame 628, a light source 630 (illustrated in phantom), and a detector 634 (illustrated in phantom) that are substantially similar to the elements illustrated and described above. Accordingly, such elements will not be described in substantial detail herein. Additionally, the detector 634 can again be utilized to capture images of the sample 620 that can be transferred as signals 615, e.g., image signals, to the analyzer 516 for purposes of analysis.

As above, the light source 630 generates and/or emits the light beam 632 (shown partially in phantom) that is directed toward the sample 620, with the light beam 632 again being used to spectroscopically scan the sample 620 for purposes of analysis. Moreover, in certain embodiments, the light source 630 can again utilize tunable laser radiation to spectroscopically interrogate the sample 620 in order to analyze and identify the physiological parameters that are present in the sample 620. For example, in some embodiments, the light source 630 can again be an MIR light source that can be selectively tuned so as to generate and/or emit a narrow linewidth, accurately settable MIR beam as the light beam 632. In one such embodiment, the light source 630 can be a quantum cascade laser (QCL), which includes a Quantum Cascade (QC) gain medium 636 that directly emits the light beam 632 that is in the mid-wavelength infrared range without any frequency conversion. Alternatively, the light source 630 can include an interband-cascade (IC) laser, a diode laser, or any other laser capable of generating radiation in the appropriate mid-wavelength infrared spectral region. Still alternatively, the light source 630 can be another suitable light source that generates and/or emits an alternatively suitable light beam.

As provided above, in one embodiment, the sampler 618 is an ATR window, or ATR crystal, that directly contacts the sweat 620 of the person 510. Attenuated Total Reflectance (ATR) is a sampling technique that can be used in conjunction with infrared spectroscopy, which enables samples to be examined directly in the solid or liquid state without further preparation. ATR uses a property of total internal reflection resulting in an evanescent wave. As utilized in the present application, the light beam 632, i.e. the mid-infrared light beam, is passed through the ATR window 618 in such a way that it reflects at least once off an inner surface 648 (or edge) of the ATR window 618 that is in contact with the sample 620. This reflection forms the evanescent wave which extends into the sample 620. The depth to which the evanescent wave extends into the sample 620 is generally determined by the wavelength of the light beam 632, the angle of incidence and the indices of refraction for the ATR window 618, and the particular components of the sample 620 being analyzed. The number of reflections may also be varied by varying the angle of incidence and the indices of refraction for the ATR window 618.

The evanescent effect as discussed above only works if the ATR window 618 is made of an optical material with a higher refractive index than the sample 620 being studied. In certain non-exclusive alternative embodiments, the materials utilized for the ATR window 618 can include germanium, KRS-5, zinc selenide, or other appropriate materials. Additionally, the shape of the ATR window 618 can depend on the type of light source 630 being utilized, and the nature of the sample 620 itself. For example, in one non-exclusive alternative embodiment, the ATR window 618 can be a rectangular slab with an outer edge 650, i.e. the edge of the ATR window 618 away from the sample 620 and nearer the light source 630, that is non-planar, e.g., rough, angled or chamfered. Stated in another manner, the ATR window 618 is positioned relative to the sample 620 such that the non-planar, outer edge 650 is spaced apart from the sample 620.

As with the previous embodiments, the light source 630 can generate and/or emit the light beam 632 that can be used to spectroscopically scan the sample 620 that has been collected and/or contacted by the sampler 618 for purposes of analysis. More specifically, as the light beam 632 scans the sample 620, via the ATR window 618, the reflected light can be monitored and an image or other suitable signal 615 can be generated that is sensed and/or captured by the image sensor 634. The captured image and/or signal 615 as sensed by the image sensor 634 captures, displays and/or provides evidence of the selected indicators or physiological parameters that are present in the sweat sample 620 of the person 510 during the specified period of time. In particular, as with the previous embodiments, depending on the specific wavelength of the light beam 632, the light beam 632 will react with the sample 620, via the ATR window 618 in this embodiment, to make different physiological parameters more apparent and/or identifiable within the image of the sample 620. Subsequently, the generated images or signals 615 can be wirelessly sent to the analyzer 516 so that the desired analysis, i.e. the desired determination and correlation of specified physiological parameters, can be undertaken.

Additionally, as with the previous embodiments, it should be appreciated that the light beam 632 can be utilized for purposes of spectroscopic analysis of the sample 620 in a single-pass or multi-pass through the sampler 618, and such use of the light beam 632 is not limited to the specific usage shown in the schematic illustration of FIG. 6.

It is understood that although a number of different embodiments of the analysis assembly 12 and methods for manufacture have been illustrated and described herein, one or more features of any one embodiment can be combined with one or more features of one or more of the other embodiment, provided that such combination satisfies the intent of the present invention. Additionally, it will be obvious to those recently skilled in the art that modifications to the analysis assembly 12 and methods of manufacture disclosed herein may occur, including substitution of various component values or modes of connection, without departing from the true spirit and scope of the disclosure.

While a number of exemplary aspects and embodiments of an analysis assembly 12 have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. An analysis assembly for analyzing one or more physiological parameters of a person during a specified period of time, the analysis assembly comprising:
a sensor assembly that senses the one or more physiological parameters of the person, the sensor assembly including (i) a sampler that collects a sample from the person during the specified period of time; and (ii) a signal generating apparatus that directs a mid-infrared light beam toward the sample and performs spectroscopy on the sample to generate a signal that is based at least in part on the one or more physiological parameters of the person, each of the sampler and the signal generating apparatus being positioned less than approximately one meter from the person during the specified period of time; and
an analyzer that receives the signal from the sensor assembly, the analyzer analyzing the signal to determine the presence of the one or more physiological parameters in the sample.

2. The analysis assembly of claim 1 wherein the sample comprises a breath sample from the person.

3. The analysis assembly of claim 1 wherein the sensor assembly is selectively coupled to the person, and wherein the sampler is selectively positioned within less than approximately twenty centimeters from a mouth of the person.

4. The analysis assembly of claim 3 further comprising a headset that is selectively coupled to the person, the headset including a coupling member that couples the headset to a head of the person, and an extension arm that is connected and extends away from the coupling member; and wherein the sampler and the signal generating apparatus are attached to the extension arm, each of the sampler and the signal generating apparatus being positioned within less than approximately twenty centimeters from the mouth of the person.

5. The analysis assembly of claim 1 wherein the sampler includes a sampler body, an intake that is coupled to the sampler body, and a pump that pumps the sample into the sampler body via the intake.

6. The analysis assembly of claim 1 wherein the signal generating apparatus includes a light source that emits the mid-infrared light beam that is directed toward the sample, and wherein the mid-infrared light beam spectroscopically scans the sample to generate the signal.

7. The analysis assembly of claim 6 wherein the light source is selectively adjustable to alternatively emit a first mid-infrared light beam having a first wavelength and a second mid-infrared light beam having a second wavelength that is different than the first wavelength.

8. The analysis assembly of claim 6 wherein the light source is a quantum cascade laser that emits the mid-infrared light beam.

9. The analysis assembly of claim 1 wherein the analyzer analyzes the signal to determine the presence of a first physiological parameter, and a second physiological parameter that is different than the first physiological parameter, and wherein the analyzer determines a ratio of the first physiological parameter to the second physiological parameter.

10. The analysis assembly of claim 9 wherein the first physiological parameter includes a ketone, and wherein the second physiological parameter includes carbon dioxide.

11. The analysis assembly of claim 1 wherein the sensor assembly is coupled to an exercise apparatus.

12. The analysis assembly of claim 1 wherein the sample comprises a sweat sample from the person.

13. The analysis assembly of claim 12 wherein the sampler is an Attenuated Total Reflectance window that is adapted to be positioned in contact with the skin of the person to collect the sweat sample of the person, and wherein the signal generating apparatus performs spectroscopy on the sweat sample to generate the signal.

14. An analysis assembly for analyzing one or more physiological parameters of a person utilizing an exercise apparatus during a specified period of time, the analysis assembly comprising:
a sensor assembly that senses the one or more physiological parameters of the person, the sensor assembly including (i) a sampler that collects a sample from the person during the specified period of time; and (ii) a signal generating apparatus that directs a mid-infrared light beam toward the sample and performs spectroscopy on the sample to generate a signal that is based at least in part on the one or more physiological parameters of the person, the sensor assembly being coupled to the exercise apparatus; and
an analyzer that receives the signal from the sensor assembly, the analyzer analyzing the signal to determine the presence of the one or more physiological parameters in the sample.

15. The analysis assembly of claim 14 wherein the analyzer analyzes the signal to determine the presence of a first physiological parameter, and a second physiological parameter that is different than the first physiological parameter, and wherein the analyzer determines a ratio of the first physiological parameter to the second physiological parameter.

16. The analysis assembly of claim 15 wherein the first physiological parameter includes a ketone, and wherein the second physiological parameter includes carbon dioxide.

17. The analysis assembly of claim 14 further comprising an exercise apparatus having a flexible, extension arm that is selectively positionable relative to the person utilizing the exercise apparatus; wherein the sampler and the signal generating apparatus are positioned at a distal end of the extension arm, each of the sampler and the signal generating apparatus being selectively positionable within less than approximately one meter from a mouth of the person.

18. An analysis assembly for analyzing one or more physiological parameters of a person during a specified period of time, the analysis assembly comprising:
a sensor assembly that senses the one or more physiological parameters of the person, the sensor assembly including (i) a sampler that collects a sweat sample from the person during the specified period of time, the sampler including an Attenuated Total Reflectance window having an inner surface that is positioned in contact with the skin of the person and contacts the sample, and a non-planar, outer edge that is spaced apart from the sample; and (ii) a signal generating apparatus that directs a mid-infrared light beam toward the sample and performs spectroscopy on the sample to generate a signal that is based at least in part on the one or more physiological parameters of the person; and
an analyzer that receives the signal from the sensor assembly, the analyzer analyzing the signal to determine the presence of the one or more physiological parameters in the sample.

19. The analysis assembly of claim 18 wherein the Attenuated Total Reflectance window is formed from a material having a window refractive index that is higher than a sample refractive index of the sample.

20. The analysis assembly of claim 18 wherein the analyzer analyzes the signal for the presence of one or more of sodium, potassium, calcium, magnesium, lactate and urea.
